(19) United States
(12) Patent Application Publication
(10) Pub. No.: US 2016/0008358 A1
(43) Pub. Date: Jan. 14, 2016

(54) STABLE PHARMACEUTICAL INJECTABLE COMPOSITIONS OF VORICONAZOLE

(71) Applicant: CADILA HEALTHCARE LIMITED, Ahmedabad (IN)

(72) Inventors: Sushrut Krishnaji Kulkarni, Ahmedabad (IN); Debjani Manoj Singh, Ahmedabad (IN); Tushar Surajmal Nahata, Ahmedabad (IN); Jaimin Mahendrrabhai Patel, Ahmedabad (IN)

(21) Appl. No.: 14/795,045

(22) Filed: Jul. 9, 2015

(30) Foreign Application Priority Data
Jul. 9, 2014 (IN) 2232/MUM/2014

(51) Int. Cl. A61K 31/506; A61K 9/08; A61K 47/02; A61K 47/48

(52) U.S. Cl. CPC A61K 31/506; A61K 47/48969; A61K 9/08; A61K 47/02

(57) ABSTRACT

The present invention relates to stable aqueous pharmaceutical compositions of voriconazole or a pharmaceutically acceptable salt thereof suitable for parenteral administration. In particular, the invention relates to ready-to-use stable injectable compositions of voriconazole or a pharmaceutically acceptable salt thereof. The invention also relates to processes for the preparation of such compositions and use thereof for the treatment of fungal infections.

### FIELD OF THE INVENTION

The present invention relates to stable aqueous pharmaceutical compositions of voriconazole or a pharmaceutically acceptable salt thereof suitable for parenteral administration. In particular, the invention relates to ready to use stable injectable compositions of voriconazole or a pharmaceutically acceptable salt thereof. The invention also relates to processes for the preparation of such compositions and use thereof for the treatment of fungal infections.

### BACKGROUND OF THE INVENTION

Voriconazole is a triazole antifungal agent, disclosed in European Patent Application 044072. It has the following structure.

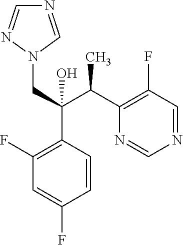 [SMILES: C[C@@H](c1ncncc1F)[C@](O)(Cn1cncn1)c1ccc(F)cc1F]

Voriconazole is designated chemically as (2R,3S)-2-(2, 4-difluorophenyl)-3-(5-fluoro-4-pyrimidinyl)-1-(1H-1,2,4triazol-1-yl)-2-butanol. Voriconazole is useful in the treatment of fungal infections particularly, Invasive aspergillosis,

Candidemia (nonneutropenics) and disseminated candidiasis, Esophageal candidiasis and Serious infections caused by Scedosporium apiospermum and Fusarium spp. including Fusarium solani. Voriconazole is a weak base and has a low aqueous solubility (0.61 mg/ml at a pH 7; 0.2 mg/ml at a pH 3) and unstable in water.

Voriconazole is susceptible to alkaline and thermal degradation and therefore, it is necessary to maintain the pH of an aqueous solution, which prevents the formation of impurities. Thus the development of aqueous intravenous solution with sufficient shelf-life is difficult.

Degradation of voriconazole results in formation of following impurities.

### Impurity A: 1-(2, 4-difluorophenyI)-2-(1H-1, 2, 4-triazol-1-yl)ethanone

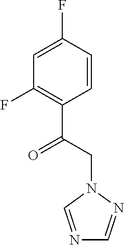 [SMILES: O=C(Cn1cncn1)c1ccc(F)cc1F]

### Impurity B: (2RS,3SR)-2-(2, 4-difluorophenyl)-3-pyrimidin-4-yl-1-(1H-1,2,4-triazol-1-yl)butan-2-ol

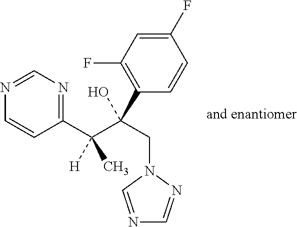 [SMILES: C[C@@H](c1ccncn1)[C@](O)(Cn1cncn1)c1ccc(F)cc1F]

### Impurity C: 4-ethyl-5-fluoropyrimidine

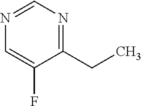

### Impurity D: (2S,3R)-2-(2,4-difluorophenyl)-3-(5-fluoropyrimidin-4-yl)-1-1](1H-1,2,4-triazol-1-yl)butan-2-ol(voriconazole enantiomer)

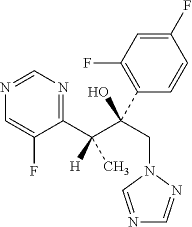 [SMILES: C[C@H](c1ncncc1F)[C@@](O)(Cn1cncn1)c1ccc(F)cc1F]

### Impurity E: [(1R,4S)-7,7-dimethyl-2-oxobicyclo[2.2.1]hept-1-yl]methane sulfonic acid((±)-10-camphorsulfonic acid)

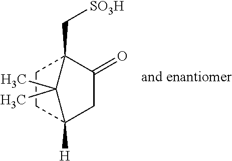

U.S. Pat. No. 5,773,443 discloses triazole derivatives which have antifungal activity, more particularly to 2-aryl-3-(3-halopyridin-4-yl or 5-halopyrimidin-4-yl)-1-(1H-1,2,4-triazol-1-yl)alkan-2-ol derivatives which are useful in the treatment of fungal infections in animals, including human beings.

U.S. Pat. No. 5,116,844 discloses novel triazole derivatives which have antifungal activity and are useful in the treatment of fungal infections in animals, including humans.

European Application No. 1001813 discloses the pharmaceutical composition of voriconazole with cyclodextrin derivatives prepared by lyophilization process. This composition should be reconstituted prior to use and exhibits enhanced stability and solubility.

European Application No. 2018866 discloses a pharmaceutical composition of voriconazole for improving the solubility of voriconazole in aqueous solution with beta-cyclodextrin. The aqueous solution is formed with a particular molar ratio of voriconazole and beta-cyclodextrin and lyophilization is used as an additional step during manufacturing process. The composition has shown stability of voriconazole upto 6 hours and is silent about long-term stability of voriconazole.

Currently, approved product of voriconazole injection (Vfend®) in market is a lyophilized sterile powder for solution for infusion. It is to be reconstituted and diluted prior to administration. The product contains voriconazole and sulfobutylether beta-cyclodextrin sodium (SBECD). By forming an inclusion complex with SBECD, the solubility and stability of voriconazole are enhanced. Because of the stability limitations of voriconazole, a ready to use aqueous solution of voriconazole is not feasible and therefore a lyophilized powder for infusion has been developed that is manufactured using an aseptic manufacturing process employing a microbial retentive filter.

U.S. Pat. No. 6,632,803 discloses lyophilized pharmaceutical composition comprising voriconazole, or a pharmaceutically acceptable derivative thereof, and a sulfobutylether beta-cyclodextrin.

All of the above documents/references have used lyophilization process to stabilize voriconazole in injectable compositions.

It is known that lyophilization process may have a considerable effect on the degradation of the pharmaceutical active ingredients in a composition, as well as a strong impact on their stability in freeze-dried form. The various variables which affect these parameters are mainly the pH, the quantity of salts present, the type and quantity of excipients in the composition, the type of cryoprotectant chosen, as well as the temperature, pressure and the time chosen for the freezing, sublimation and drying operations. These different variables influence the physical state of the freeze-dried product obtained, namely: vitreous amorphous, soft amorphous, crystalline or a combination of these states. Also, lyophilization is very complex and expensive process for stabilization, thus there exists an alternative need to stabilize the composition without using this process.

Hence, there still remains a need for alternate stable ready to use pharmaceutical compositions of voriconazole suitable for parenteral administration.

### SUMMARY OF THE INVENTION

In one general aspect, there is provided a stable aqueous pharmaceutical composition suitable for parenteral administration comprising voriconazole or a pharmaceutically acceptable salt thereof and one or more pharmaceutically acceptable excipients.

In another general aspect, there is provided a ready to use solution of voriconazole or a pharmaceutically acceptable salt thereof for parenteral administration.

In another general aspect, there is provided a stable aqueous pharmaceutical composition suitable for parenteral administration comprising voriconazole or a pharmaceutically acceptable salt thereof and one or more pharmaceutically acceptable excipients, wherein the composition is in the form of ready to use solution and is stored under refrigerated conditions at a temperature of about 2° C. to about 8° C.

In another general aspect, there is provided a stable aqueous pharmaceutical composition suitable for parenteral administration comprising voriconazole or a pharmaceutically acceptable salt thereof and one or more pharmaceutically acceptable excipients, wherein the composition is not a lyophilized product.

In another general aspect, there is provided a stable aqueous pharmaceutical composition comprising voriconazole or a pharmaceutically acceptable salt thereof, wherein the composition retains at least about 90% of the potency of voriconazole in the pharmaceutical composition after storage for twelve months under refrigerated conditions at a temperature of about 2° C. to about 8° C.

In another general aspect, there is provided a stable aqueous pharmaceutical composition comprising voriconazole or a pharmaceutically acceptable salt thereof, sulfobutylether beta-cyclodextrin and 1-(2, 4-difluorophenyl)-2-(1H-1,2,4-triazol-1-yl)ethanone in an amount greater than zero and less than about 0.5% as measured by HPLC after storage for twelve months under refrigerated conditions at a temperature of about 2° C. to about 8° C.

In another general aspect, there is provided a stable aqueous pharmaceutical composition comprising 10 mg/ml of voriconazole or a pharmaceutically acceptable salt thereof and 160 mg/ml of sulfobutylether beta-cyclodextrin, wherein the composition retains at least about 90% of the potency of voriconazole under refrigerated condition (about 2° C. to about 8° C.) for at least twelve months.

In another general aspect, there is provided a stable aqueous pharmaceutical composition comprising 10 mg/ml of voriconazole or a pharmaceutically acceptable salt thereof and 160 mg/ml of sulfobutylether beta-cyclodextrin wherein the composition contains not more than 1% total degradation impurities as measured by HPLC after storage for twelve months under refrigerated conditions at a temperature of about 2° C. to about 8° C.

In another general aspect, there is provided a stable aqueous pharmaceutical composition of voriconazole suitable for parenteral administration is prepared by a process comprising:

a) preparing a solution of voriconazole and sulfobutylether beta-cyclodextrin in water;

b) adjusting the final volume with water;

c) sparging the solution of step b) with pharmaceutically inert gas; and

d) sterilizing the solution of step c) and, filling the resulting solution into the suitable container with headspace of pharmaceutically inert gas.

In another general aspect, there is provided a method for the treatment of fungal infection comprising parenterally administering a stable aqueous pharmaceutical composition comprising voriconazole and one or more pharmaceutically acceptable excipients, wherein the composition is stabilized by an inert gas and the composition is in the form of a ready to use solution.

The details of one or more embodiments of the present invention are set forth in the description below. Other features, objects and advantages of the invention will be apparent from the description.

### DETAILED DESCRIPTION OF THE INVENTION:

The inventors of the present invention have surprisingly found that it is possible to develop a stable ready to use composition of voriconazole suitable for parenteral administration.

The inventors of the present invention have found that stability of the injectable compositions of voriconazole was improved upon storing the composition at refrigerated condition (about 2° C. to about 8° C.) and removing the dissolved oxygen from the bulk solution. Stability may be further improved by filling the composition in airtight container having a headspace volume occupied by an inert gas. Removal of dissolved oxygen may be readily accomplished by bubbling an inert gas, preferably nitrogen.

The inventors of the present invention have surprisingly found that the liquid composition of voriconazole can be produced which remains stable for at least one year at about 2° C. to about 8° C. without the need of lyophilization process.

The term ‘voriconazole’ used throughout the specification refers to voriconazole per se, pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs thereof, its various crystalline or amorphous forms or mixtures thereof.

The term “stable” refers to both the physical and chemical stability of a composition in any form, such as a solution. A composition is said to be stable if it exhibits minimal change over time relative to when it is manufactured. Stability is measured at various time points through a planned product expiration date with evaluation criteria including such items as therapeutic activity, appearance, levels of particulate matter, pH, content of active ingredient(s), and levels of degradation products, impurities, or related substances.

The term “suitable for parenteral administration”, as used herein with respect to the mode of administration, includes all modes of administration other than oral ingestion. Examples of “parenteral” administration include intravenous, subcutaneous or intramuscular administration where the voriconazole composition is generally a solution that is formulated to enter the body of the patient either through an injection via syringe or intravenously, through an IV tube, or other invasive modes known to the artisan.

The term “inert gas” refers to a gas which expels or removes the dissolved oxygen content from the composition and maintains inert gaseous atmospheric environment in the composition. Examples of inert gas include nitrogen or carbon dioxide.

According to present invention, the composition of the present invention is in a form selected from solution, suspension, or emulsion suitable for parenteral administration comprising voriconazole or a pharmaceutically acceptable salt thereof with one or more parenterally acceptable excipients.

It is preferred that compositions of the present invention are provided in ready to use solution. It will be readily appreciated by those skilled in the art how to administer compositions of the present invention to a human or an animal.

In one embodiment, there is provided a stable aqueous pharmaceutical composition suitable for parenteral administration comprising voriconazole and one or more pharmaceutically acceptable excipients.

In another embodiment, there is provided a ready to use injectable composition of voriconazole having less than 1% of total voriconazole degradation impurities.

In another embodiment, there is provided ready to use injectable composition of voriconazole having less than 0.5% each of the voriconazole degradation impurity A, impurity B, impurity C, impurity D and impurity E.

In another embodiment, the composition of the present invention comprises voriconazole in an amount in the range from about 5 mg/ml to about 100 mg/ml, preferably about 5 mg/ml to about 50 mg/ml, more preferably from about 10 mg/ml to about 20 mg/ml.

In another embodiment, the composition of the present invention comprises one or more pharmaceutically acceptable excipients selected from the group consisting of solubilizing aids, surfactants, vehicles, co-solvents, pH adjusting agents, tonicity adjusting agents, buffers, diluents, chelating agents, antioxidants, preservatives, or combination thereof.

The term ‘solubilizing aid’ is meant for any compound that may assist in solubilizing the active by accommodating the active in a cavity formed in the solubilizing aid to form inclusion complexes.

Suitable solubilizing aids, which may be used in the present invention include, but are not limited to cyclodextrins such as α-cyclodextrin, β-cyclodextrin, γ-cyclodextrin and the like as well as their pharmaceutically useful derivatives. Combinations of more than one of these solubilizing aids in different ratios or proportions as required are covered within the scope of the invention without limitation.

Preferably used cyclodextrin may be β-cyclodextrin. The β-cyclodextrin derivative/s comprising one or more of hydroxyl alkylated β-cyclodextrins, sulfoalkyl ether β-cyclodextrin, branched β-cyclodextrins, methylated β-cyclodextrins, ethylated β-cyclodextrins, and anionic β-cyclodextrins. The amount of cyclodextrins may be in the range from about 1% w/v to about 50% w/v, preferably from about 1% w/v to about 30% w/v, more preferably from about 10% w/v to about 20% w/v of the composition.

According to the present invention, the composition comprises voriconazole and cyclodextrin derivative in the molar ratio in a range of from about 1:1 to about 1:10.

In another embodiment of the present invention, there is provided a stable aqueous pharmaceutical composition suitable for parenteral administration comprising voriconazole or a pharmaceutically acceptable salt thereof, a cyclodextrin derivative, wherein the composition is sparged with an inert gas.

In another embodiment, there is provided a stable aqueous pharmaceutical composition suitable for parenteral administration comprising voriconazole or a pharmaceutically acceptable salt thereof and a cyclodextrin derivative, wherein the composition is sealed in airtight container comprising a fill volume of the composition and a headspace volume occupied by an inert gas.

According to present invention, the pharmaceutically inert gas may be bubbled into the solution to drive out oxygen, which may be selected from nitrogen or carbon dioxide.

Suitable air tight containers used to accommodate the voriconazole composition may be any container suitable for parenteral composition those are known to the person skilled in the art. Examples of such air tight containers are vials, ampules, bottles, prefilled syringes etc.

In another embodiment, a stable aqueous pharmaceutical composition suitable for parenteral administration comprising voriconazole or a pharmaceutically acceptable salt thereof and one or more pharmaceutically acceptable parenteral excipients, wherein the composition retains at least 90% of the potency of voriconazole at refrigerated condition (about 2° C. to about 8° C.) for at least about one, two, three, six or twelve months.

In another embodiment, a stable aqueous pharmaceutical composition suitable for parenteral administration comprising 10 mg/ml of voriconazole or a pharmaceutically acceptable salt thereof and 160 mg/ml of sulfobutylether beta-cyclodextrin, wherein the composition retains at least about 90% of the potency of voriconazole in the pharmaceutical composition after storage for twelve months under refrigerated conditions at a temperature of about 2° C. to about 8° C.

In another embodiment, a stable aqueous pharmaceutical composition suitable for parenteral administration comprising 10 mg/ml of voriconazole or a pharmaceutically acceptable salt thereof and 160 mg/ml of sulfobutylether beta-cyclodextrin, wherein the composition retains at least about 95% of the potency of voriconazole in the pharmaceutical composition after storage for twelve months under refrigerated conditions at a temperature of about 2° C. to about 8° C.

In another embodiment, a stable aqueous pharmaceutical composition suitable for parenteral administration comprising 10 mg/ml of voriconazole or a pharmaceutically acceptable salt thereof and 160 mg/ml of sulfobutylether beta-cyclodextrin, wherein the composition retains at least about 95% of the potency of voriconazole in the pharmaceutical composition after storage for twelve months under refrigerated conditions at a temperature of about 2° C. to about 8° C., and wherein the composition is sealed in airtight container comprising a fill volume of the composition and a headspace volume occupied by an inert gas.

In another embodiment, a stable aqueous pharmaceutical composition suitable for parenteral administration comprising 10 mg/ml of voriconazole or a pharmaceutically acceptable salt thereof and 160 mg/ml of sulfobutylether beta-cyclodextrin, wherein the composition contains less than 0.5% each of the voriconazole degradation impurity A, impurity B and impurity C, impurity D and impurity E as measured by HPLC after storage for twelve months under refrigerated conditions at a temperature of about 2° C. to about 8° C.

In another embodiment, a stable aqueous pharmaceutical composition suitable for parenteral administration comprising voriconazole or a pharmaceutically acceptable salt thereof, sulfobutylether beta-cyclodextrin and 4-ethyl-5-fluoropyrimidine in an amount greater than zero and less than 0.5% as measured by HPLC after storage for twelve months under refrigerated conditions at a temperature of about 2° C. to about 8° C.

In another embodiment, a stable aqueous pharmaceutical composition suitable for parenteral administration comprising voriconazole or a pharmaceutically acceptable salt thereof, sulfobutylether beta-cyclodextrin and 1-(2, 4-difluorophenyl)-2-(1H-1,2,4-triazol-1-yl) ethanone in an amount greater than zero and less than 0.5% as measured by HPLC after storage for twelve months under refrigerated conditions at a temperature of about 2° C. to about 8° C.

In another embodiment, a stable aqueous pharmaceutical composition suitable for parenteral administration comprising 10 mg/ml of voriconazole or a pharmaceutically acceptable salt thereof and 160 mg/ml of sulfobutylether beta-cyclodextrin, wherein the composition contains not more than 1% total degradation impurity as measured by HPLC after storage for twelve months under refrigerated conditions at a temperature of about 2° C. to about 8° C.

Potency herein corresponds to an amount of active ingredient (Voriconazole) present in the composition. Potency of voriconazole is determined by using assay procedure described in the U.S. pharmacopoeia.

Suitable surfactants which may be used in the present invention include, but not limited to polysorbates, their ether ethoxylates, produced by reaction of sorbitan esters with ethylene oxide, polyoxyethylene alkyl phenol, polyoxyethylene cetyl ether, polyoxyethylene alkyl-aryl ether, polyoxyethylene monolaurate, polyoxyethylene vegetable oil, polyoxyethylene sorbitan monolaurate, polyoxyethylene esters or mixed fatty and resin acids, polyoxyethylene sorbitol lanolin derivative, polyoxyethylene tridecylether, polyoxyethylene sorbitan esters of mixed fatty and resin acids, polyoxyethylene sorbitan monostearate, polyoxyethylene sorbitan monooleate, polyoxyethylene monostearate, polyoxyethylene stearyl ether, polyoxyethylene oleyl ether, polyoxyethylene tridecyl ether, polyoxyethylene fatty alcohol, polyoxyethylene alkyl amine, polyoxyethylene glycol monopalmitate, polyoxyethylene sorbitan monopalmitate, polyoxyethylene cetyl ether, polyoxyethylene oxypropylene stearate, polyoxyethylene lauryl ether, polyoxyethylene lanolin derivative, sodium oleate, quaternary ammonium derivative, potassium oleate, N-cetyl N-ethyl morpholinium ethosulfate, sodium lauryl sulfate or mixtures thereof.

Suitable vehicles and co-solvents which may be used in the present invention include, but not limited to one or more of water and its various grades suitable for parenteral administration such as water for injection, bacteriostatic water for injection, sterile, aqueous solutions of electrolytes and/or dextrose; alcohols such as ethanol, isopropanol; polyols such as propylene glycol, polyethylene glycol, glycerol; dimethyl sulfoxide (DMSO); dimethyl acetamide (DMAC); 3-dimethyl-2-imidazolidinone (DMI); N-Methyl-2-Pyrrolidone (M-PYROL); and vegetable oils. The amount of vehicle and co-solvents may be in the range from about 0.01% w/v to about 95% w/v of the composition.

Suitable pH adjusting agents which may be used in the present invention include, but not limited to sodium hydroxide, hydrochloric acid, citric acid, acetic acid, fumaric acid, hydrochloric acid, malic acid, nitric acid, phosphoric acid, propionic acid, sulfuric acid, tartaric acid, or combinations thereof.

The amount of pH-adjusting agent may vary depending on the desired pH of the composition and the amount of voriconazole in the solution; can be determined by one of ordinary skill in the art. For example, in general, the amount of a pH-adjusting agent, such as sodium hydroxide, in compositions of the present invention will directly vary depending on the desired concentration of the voriconazole. The exact amount of pH-adjusting agent to be employed will depend on the particular agent and upon the buffering capacity of the aqueous medium and other components of the composition employed.

Compositions of the present invention suitable for parenteral administration need very few constituents, but it is generally preferred that an injectable solution is made up with saline to provide a solution which is iso-osmotic with blood. The osmolality can be set by variation of the amounts of the dissolved substances present in the composition besides voriconazole and any further substances present, and/or by addition of a tonicity adjusting agent.

Suitable tonicity adjusting agent which may be used in the present invention include, but not limited to physiologically tolerated salt, such as, for example, sodium chloride or potassium chloride, or a physiologically tolerated polyol, such as, for example, a sugar alcohol, in particular sorbitol or glycerol, in the concentration necessary for adjusting tonicity.

Suitable buffers which may be used in the present invention include, but not limited to borate buffers, tartarate buffers, lactate buffers, citrate buffers, phosphate buffers, citric acid/phosphate buffers, carbonate/carbonic acid buffers, succinate/succinic acid buffers, ammonium buffers, and tris(hydroxymethyl)aminomethane/hydrochloric acid buffers and the like. The amount of buffer may range from about 0.01% w/v to about 15% w/v of the composition.

Suitable diluents which may be used in the present invention include, but not limited to a mannitol, glycine, lactose, sucrose, trehalose, dextran, hydroxyethyl starch, ficoll or gelatin and the like. The amount of diluents may range from about 0.01% w/v to about 90% w/v of the composition.

Suitable chelating agents which may be used in the present invention include, but not limited to edetate disodium (EDTA); edetate trisodium; edetate tetrasodium; and diethyleneamine pentaacetate, preferably EDTA. The amount of the chelating agent present in the aqueous pharmaceutical composition may range from about 0.001-2.0% w/v of the composition.

Suitable antioxidants which may be used in the present invention include, but not limited to monothioglycerol, ascorbic acid derivative, butylated hydroxy anisole, butylated hydroxy toluene, alkyl gallate, sodium meta-bisulfite, sodium bisulfite, sodium dithionite, sodium thioglycollic acid, sodium formaldehyde sulfoxylate, tocopherol and derivatives thereof, and sodium sulfite. The most preferred antioxidant is monothioglycerol. The amount of antioxidants may range from about 0.01% w/v to about 10.0% w/v of the composition.

Suitable preservative which may be used in the present invention include, but not limited to phenol, m-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, or mixtures thereof. The amount of preservative may range from about 0.01% w/v to about 10.0% w/v of the composition.

In another embodiment, a stable aqueous pharmaceutical composition of voriconazole suitable for parenteral administration is prepared by a process comprising:

a) preparing a solution of voriconazole or a pharmaceutically acceptable salt thereof and one or more pharmaceutically acceptable excipients in suitable vehicle;

b) adjusting the pH of solution with one or more pH adjusting agents and making final volume with a suitable vehicle;

c) sparging the solution of step b) with pharmaceutically inert gas; and

d) sterilizing the solution of step c) and, filling the resulting solution into the suitable container with headspace of pharmaceutically inert gas.

In another embodiment, a stable aqueous pharmaceutical composition of voriconazole suitable for parenteral administration is prepared by a process comprising:

a) preparing a solution of voriconazole and sulfobutylether beta-cyclodextrin in water;

b) making final volume with water;

c) sparging the solution of step b) with pharmaceutically inert gas; and

d) sterilizing the solution of step c) and, filling the resulting solution into the suitable container with headspace of pharmaceutically inert gas.

The voriconazole composition may be sterilized in order to comply with regulatory standards and also to ensure safe delivery of voriconazole in therapeutic effective concentration. Sterilization renders the composition free from viable pathogens and ensures a low endotoxin level in compliance with regulatory standards. Sterilization may be accomplished by conventional methods and techniques. For example, solution compositions may be sterilized by sterile filtration, irradiation, heating, or autoclaving of the solution. Other liquid compositions, such as suspensions, emulsions, and the like, may also be sterilized by conventional methods including, without limitation, irradiation, heating, autoclaving, or a combination thereof.

The invention further provides a method of treating fungal infections in patient comprising administering to the patient a pharmaceutical composition comprising voriconazole or a pharmaceutically acceptable salt thereof and one or more pharmaceutically acceptable excipients.

More particularly the fungal infections include, Invasive aspergillosis, Candidemia (nonneutropenics) and disseminated candidiasis, Esophageal candidiasis and Serious infections caused by Scedosporium apiospermum and Fusarium spp. including Fusarium solani.

The invention is further illustrated by the following examples which are provided to be exemplary of the invention and do not limit the scope of the invention.

### EXAMPLE 1

TABLE 1

| Sr. | | Quantity |
|---|---|---|
| No. | Ingredients | (mg/ml) |
| 1. | Voriconazole | 10.0 |
| 2. | Sulfobutyl ether beta-cyclodextrin | 160 |
| | sodium | |
| 3. | Water for Injection | q.s. to 1 ml |

Procedure:

Approximately 80% of water for Injection was taken. This solution was sparged with Nitrogen to obtain dissolved oxygen level less than 2 ppm. Weighed quantity of Sulfobutyl ether beta-cyclodextrin sodium was added and stirred till a clear, colourless solution was obtained. Voriconazole was added to the solution and stirred to get a clear solution. The volume of the solution was made up to the batch size with Water for Injection and stirred for 5 min. The bulk solution was filtered through 0.22μ filter and filled with headspace of Nitrogen into glass vials.

The stability study of this composition was conducted at about 2° C. to about 8° C. over the period of twelve months. At the same time parallel study was conducted at 25° C.±2° C. for the period of six months.

The amount of impurities measured in the composition after the storage period indicated that the liquid composition was stable at about 2° C. to about 8° C.

Stability Data (at about 2° C. to about 8° C.) of Example 1

TABLE 2

| | | After 3 | After 6 | After 12 |
|---|---|---|---|---|
| Test | Initial | months | months | months |
| pH | 5.14 | 4.94 | 5.01 | 4.95 |
| Assay of Voriconazole | 100 | 99.6 | 99.4 | 99.3 |
| Impurity A | BQL | BQL | 0.06 | 0.1 |
| Unknown impurity | BQL | BQL | 0.05 | 0.09 |
| Total impurity | NIL | NIL | 0.11 | 0.19 |

BQL—Below qualification level

Stability Data (at 25° C.±2° C.) of Example 1

TABLE 3

| | | After 1 | After 3 | After 6 |
|---|---|---|---|---|
| Test | Initial | months | months | months |
| pH | 5.14 | 5.27 | 4.88 | 4.86 |
| Assay of Voriconazole | 100 | 99.25 | 98.2 | 96.4 |
| Impurity A | BQL | 0.4 | 1.0 | 1.8 |
| Unknown impurity | BQL | 0.25 | 0.85 | 1.6 |
| Total impurity | NIL | 0.65 | 1.9 | 3.4 |

BQL—Below qualification level

While the invention has been described in terms of its specific embodiments, certain modifications and equivalents will be apparent to those skilled in the art and are intended to be included within the scope of the invention.

1. A stable aqueous pharmaceutical composition suitable for parenteral administration comprising voriconazole or a pharmaceutically acceptable salt thereof, and one or more pharmaceutically acceptable excipients, wherein the composition is in the form of a ready to use solution.

2. The stable aqueous pharmaceutical composition according to claim 1, wherein the composition is stored under refrigerated conditions at a temperature of about 2° C. to about 8° C.

3. The stable aqueous pharmaceutical composition according to claim 1, wherein the composition is sparged with an inert gas.

4. The stable aqueous pharmaceutical composition according to claim 1, wherein the one or more pharmaceutically acceptable excipients comprising one or more of solubilizing aids, surfactants, vehicles, co-solvents, pH adjusting agents, tonicity adjusting agents, chelating agents, antioxidants, preservatives, stabilizers, or combination thereof.

5. The stable aqueous pharmaceutical composition according to claim 4, wherein the solubilizing aid comprises one or more of cyclodextrin derivatives such as alpha-cyclodextrin, beta-cyclodextrin, or gamma-cyclodextrin.

6. The stable aqueous pharmaceutical composition according to claim 5, wherein the composition comprises voriconazole and the cyclodextrin derivative in a molar ratio in a range from about 1:1 to about 1:10.

7. The stable aqueous pharmaceutical composition according to claim 1, wherein the composition retains at least about 90% of the potency of voriconazole in the pharmaceutical composition after storage for twelve months under refrigerated conditions at a temperature of about 2° C. to about 8° C.

8. The stable aqueous pharmaceutical composition according to claim 1, wherein the composition comprising 10 mg/ml of voriconazole or a pharmaceutically acceptable salt thereof and 160 mg/ml of sulfobutylether beta-cyclodextrin, wherein the composition retains at least about 90% of the potency of voriconazole in the pharmaceutical composition after storage for twelve months under refrigerated conditions at a temperature of about 2° C. to about 8° C.

9. The stable aqueous pharmaceutical composition according to claim 1, wherein the composition contains less than 0.5% of 1-(2,4-difluorophenyl)-2-(1H-1,2,4-triazol-1-yl) ethanone as measured by HPLC after storage for twelve months under refrigerated conditions at a temperature of about 2° C. to about 8° C.

10. The stable aqueous pharmaceutical composition according to claim 1, wherein the composition contains not more than 1% of total degradation impurities as measured by HPLC after storage for twelve months under refrigerated conditions at a temperature of about 2° C. to about 8° C.

11. The stable aqueous pharmaceutical composition of voriconazole suitable for parenteral administration according to claim 1 is prepared by a process comprising:
a) preparing a solution of voriconazole and sulfobutylether beta-cyclodextrin in water;
b) making the final volume with water;
c) sparging the solution of step b) with a pharmaceutically inert gas; and
d) sterilizing the solution of step c) and filling the resulting solution into a suitable container with headspace of pharmaceutically inert gas.

12. A method for the treatment of fungal infection comprising parenterally administering a stable aqueous pharmaceutical composition comprising voriconazole and one or more pharmaceutically acceptable excipients, wherein the composition is stabilized by using an inert gas and the composition is in the form of a ready to use solution.

13. A ready to use solution of voriconazole or a pharmaceutically acceptable salt thereof for parenteral administration.
